(19) United States
(12) Patent Application Publication
(10) Pub. No.: US 2023/0249885 A1
(43) Pub. Date: Aug. 10, 2023

(54) Hand Sanitizer Pouch Assembly

(71) Applicants: Osama Zuaiter, Madison, AL (US); Rola Zuaiter, Madison, AL (US)

(72) Inventors: Osama Zuaiter, Madison, AL (US); Rola Zuaiter, Madison, AL (US)

(21) Appl. No.: 17/668,920

(22) Filed: Feb. 10, 2022

(51) Int. Cl. B65D 75/58; B65D 5/72; B65D 83/08; A61L 2/18

(52) U.S. Cl. CPC B65D 75/5822; B65D 5/724; B65D 83/0805; A61L 2/18; A61L 2202/181

(57) ABSTRACT

A hand sanitizer pouch assembly includes a box that has a dispensing chute integrated therein. A plurality of pouches is each stored in the box. Each of the pouches is individually removable from the dispensing chute in the box and each of the pouches can be individually handled by a respective user to inhibiting contact transmission of bacteria or viruses between a plurality of users. Each of the pouches stores a fluid hand sanitizer and each of the pouches is comprised of a rippable material. In this way a respective one of the pouches can be ripped open by a user thereby facilitating the hand sanitizer to be released from the respective pouch for sanitizing the user's hands.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable
STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to hand sanitizer devices and more particularly pertains to a new hand sanitizer device for sanitizing a user's hands without requiring the user to touch an object that has previously been touched by a plurality of other users. The device includes a box and a plurality of pouches that are stored in the box. Each of the pouches contains a fluid hand sanitizer and each of the pouches can be individually removed from the box. In this way the user can dispense the hand sanitizer on their hands without the risk of contact transmission of bacteria or viruses from other users that commonly results from employing a single bottle of hand sanitizer for multiple users.

### (2) Description of Related Art including information disclosed under 37 CFR 1.97 and 1.98

The prior art relates to hand sanitizer devices including a variety of single use packets that each contain a fluid substance. In no instance does the prior art disclose a box which contains a plurality of pouches, each containing a fluid hand sanitizer, to facilitate a user to sanitize their hands without touching an object that has previously been touched by a plurality of users.

### BRIEF SUMMARY OF THE INVENTION

An embodiment of the disclosure meets the needs presented above by generally comprising a box that has a dispensing chute integrated therein. A plurality of pouches is each stored in the box. Each of the pouches is individually removable from the dispensing chute in the box and each of the pouches can be individually handled by a respective user to inhibiting contact transmission of bacteria or viruses between a plurality of users. Each of the pouches stores a fluid hand sanitizer and each of the pouches is comprised of a rippable material. In this way a respective one of the pouches can be ripped open by a user thereby facilitating the hand sanitizer to be released from the respective pouch for sanitizing the user's hands.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective in-use view of a hand sanitizer pouch assembly according to an embodiment of the disclosure.

FIG. 2 is a perspective cut-away view of an embodiment of the disclosure.

FIG. 3 is a front view of an embodiment of the disclosure.

FIG. 4 is a left side view of an embodiment of the disclosure.

FIG. 5 is a cross sectional view taken along line 5-5 of FIG. 3 of an embodiment of the disclosure.

FIG. 6 is a perspective view of an embodiment of the disclosure.

### DETAILED DESCRIPTION OF THE INVENTION

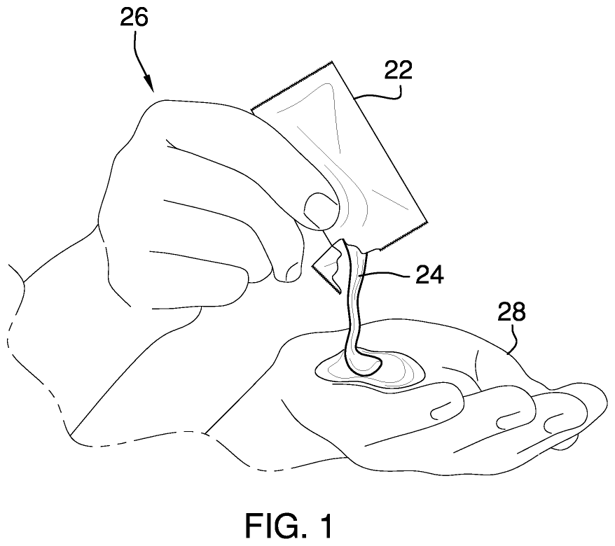

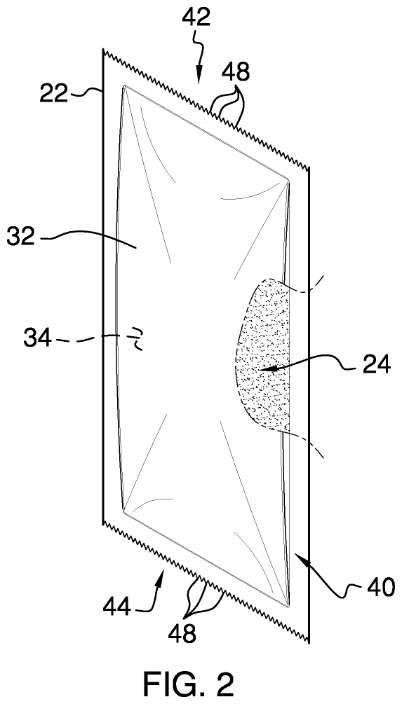

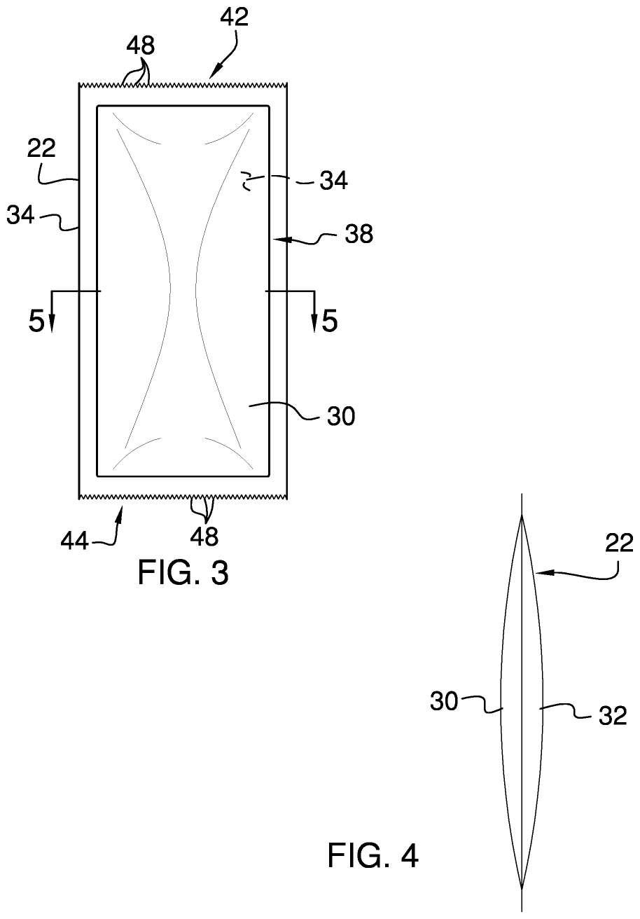

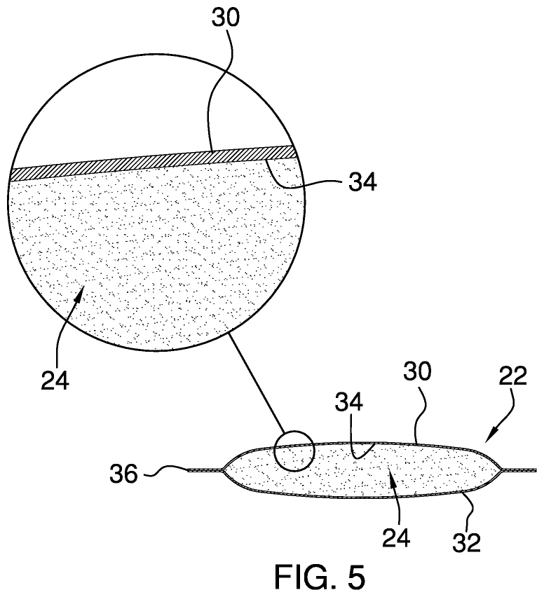

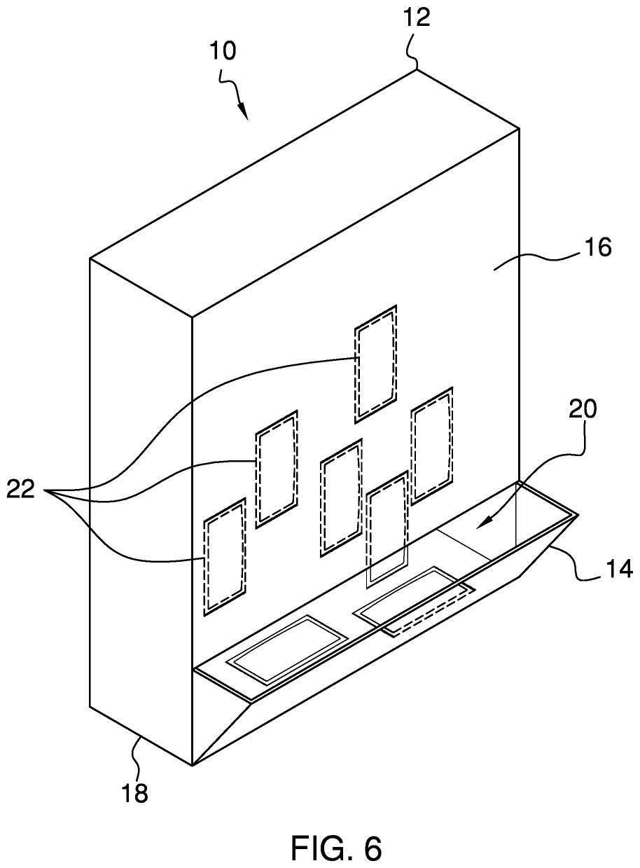

With reference now to the drawings, and in particular to FIGS. 1 through 6 thereof, a new hand sanitizer device embodying the principles and concepts of an embodiment of the disclosure and generally designated by the reference numeral 10 will be described.

As best illustrated in FIGS. 1 through 6, the hand sanitizer pouch assembly 10 generally comprises a box 12 that has a dispensing chute 14 integrated therein. The box 12 has a front wall 16 and a bottom wall 18, the front wall 16 has an opening 20 extending into an interior of the box 12 and the opening 20 is positioned adjacent to the bottom wall 18. The dispensing chute 14 angles forwardly from the front wall 16 and the dispensing chute 14 is aligned with the opening 20 in the front wall 16. The box 12 may be mounted in a public setting, such as a restaurant, a school, an office building or any other location that is typically occupied by a plurality of people.

A plurality of pouches 22 is included, each of the pouches 22 is stored in the box 12 and each of the pouches 22 is individually removable from the dispensing chute 14 in the box 12. In this way each of the pouches 22 can be individually handled by a respective user 26 thereby inhibiting contact transmission of bacteria or viruses between a plurality of users 26. Each of the pouches 22 stores a fluid hand sanitizer 24 that is approved for use on human skin. Each of the pouches 22 is comprised of a fluid impermeable material for containing the fluid hand sanitizer 24. Additionally, each of the pouches 22 is comprised of a rippable material such that a respective one of the pouches 22 can be ripped open by a user 26. In this way the hand sanitizer 24 can be released from the respective pouch 22 for sanitizing the user's hands 28. Moreover, each of the pouches 22 may contain approximately 0.14 fluid ounces of the fluid hand sanitizer 24.

Each of the pouches 22 has a first side 30 and a second side 32, and each of the first side 30 and the second side 32 has a first surface 34 and a perimeter edge 36. The first surface 34 of the first side 30 has a first bonded portion 38, and the first bonded portion 38 is coextensive with the perimeter edge 36 of the first side 30. The first surface 34 of the second side 32 has a second bonded portion 40 and the second bonded portion 40 is coextensive with the perimeter edge 36 of the second side 32. The first bonded portion 38 is bonded to the second bonded portion 40 to define a space between the first side 30 and the second side 32 for containing the fluid hand sanitizer 24.

The perimeter edge 36 of each of the first side 30 and the second side 32 has a top side 42 and a bottom side 44. The top side 42 has a plurality of ridges 46 that is distributed along a full length of the top side 42 for enhancing ripping the top side 42. The bottom side 44 has a plurality of ridges 48 that is distributed along a full length of the bottom side 44 for enhancing ripping the bottom side 44. Each of the pouches 22 is elongated between the top side 42 and the bottom side 44 of the perimeter edge 36.

In use, the user 26 removes a respective one of the pouches 22 from the dispensing chute 14 when the user 26 wishes to sanitize their hands 28. In this way the user 26 does not have a touch a bottle of hand sanitizer, for example, that will likely be touched by a variety of different users. In this way contact transmission of bacteria or viruses is inhibited that would otherwise likely occur from touching the bottle of hand sanitizer. The respective pouch 22 is torn open to facilitate the hand sanitizer 24 that is contained in the respective pouch 22 to be released onto the user's hands 28. In this way the user 26 can sanitize their hands 28 without having to touch an object that has already been touched by multiple other users.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded.

A reference to an element by the indefinite article “a” does not exclude the possibility that more than one of the element is present, unless the context clearly requires that there be only one of the elements.

I claim:

1. A hand sanitizer pouch assembly for dispensing a measured amount of hand sanitizer in a single use fashion for sanitizing a user's hands, said assembly comprising:
a box having a dispensing chute being integrated therein; and
a plurality of pouches, each of said pouches being stored in said box, each of said pouches being individually removable from said dispensing chute in said box wherein each of said pouches is configured to be individually handled by a respective user thereby inhibiting contact transmission of bacteria or viruses between a plurality of users, each of said pouches storing a fluid hand sanitizer, each of said pouches being comprised of a fluid impermeable material for containing said fluid hand sanitizer, each of said pouches being comprised of a rippable material wherein a respective one of said pouches is configured to be ripped open by a user thereby facilitating said hand sanitizer to be released from said respective pouch for sanitizing the user's hands.

2. The assembly according to claim 1, wherein said box has a front wall and a bottom wall, said front wall having an opening extending into an interior of said box, said opening being positioned adjacent to said bottom wall, said dispensing chute angling forwardly from said front wall, said dispensing chute being aligned with said opening in said front wall.

3. The assembly according to claim 1, wherein each of said pouches has a first side and a second side, each of said first side and said second side having a first surface and a perimeter edge, said first surface of said first side having a first bonded portion, said first bonded portion being coextensive with said perimeter edge of said first side, said first surface of said second side having a second bonded portion, said second bonded portion is coextensive with said perimeter edge of said second side.

4. The assembly according to claim 3, wherein said first bonded portion is bonded to said second bonded portion to define a space between said first side and said second side for containing said fluid hand sanitizer.

5. The assembly according to claim 3, wherein said perimeter edge of each of said first side and said second side has a top side and a bottom side, said top side having a plurality of ridges being distributed along a full length of said top side for enhancing ripping said top side, said bottom side having a plurality of ridges being distributed along a full length of said bottom side for enhancing ripping said bottom side.

6. A hand sanitizer pouch assembly for dispensing a measured amount of hand sanitizer in a single use fashion for sanitizing a user's hands, said assembly comprising:
a box having a dispensing chute being integrated therein, said box having a front wall and a bottom wall, said front wall having an opening extending into an interior of said box, said opening being positioned adjacent to said bottom wall, said dispensing chute angling forwardly from said front wall, said dispensing chute being aligned with said opening in said front wall; and
a plurality of pouches, each of said pouches being stored in said box, each of said pouches being individually removable from said dispensing chute in said box wherein each of said pouches is configured to be individually handled by a respective user thereby inhibiting contact transmission of bacteria or viruses between a plurality of users, each of said pouches storing a fluid hand sanitizer, each of said pouches being comprised of a fluid impermeable material for containing said fluid hand sanitizer, each of said pouches being comprised of a rippable material wherein a respective one of said pouches is configured to be ripped open by a user thereby facilitating said hand sanitizer to be released from said respective pouch for sanitizing the user's hands, each of said pouches having a first side and a second side, each of said first side and said second side having a first surface and a perimeter edge, said first surface of said first side having a first bonded portion, said first bonded portion being coextensive with said perimeter edge of said first side, said first surface of said second side having a second bonded portion, said second bonded portion being coextensive with said perimeter edge of said second side, said first bonded portion being bonded to said second bonded portion to define a space between said first side and said second side for containing said fluid hand sanitizer, said perimeter edge of each of said first side and said second side having a top side and a bottom side, said top side having a plurality of ridges being distributed along a full length of said top side for enhancing ripping said top side, said bottom side having a plurality of ridges being distributed along a full length of said bottom side for enhancing ripping said bottom side.

7. A method of sanitizing a plurality of user's hands while inhibiting contact transmission of bacteria and viruses between the users, the steps of the method comprising:
providing a box having a dispensing chute being integrated into said box;
providing a plurality of pouches, each of said pouches being positioned in said box;
providing a fluid hand sanitizer, each of said pouches containing a measured amount of said fluid hand sanitizer;
removing a respective one of said pouches from said dispensing chute in said box;
ripping said respective pouch open; and
dispensing said measured amount of said hand sanitizer in said respective pouch onto a user's hand for sanitizing the user's hand thereby inhibiting a plurality of user's from touching a common object to sanitize the user's hands thereby inhibiting contact transmission of bacteria or viruses between the users.
